(19) United States
(12) Patent Application Publication
(10) Pub. No.: US 2025/0090487 A1
(43) Pub. Date: Mar. 20, 2025

(54) Topical anesthetic agent-clay composite compositions

(71) Applicant: ANDROS PHARMACEUTICALS CO., LTD., Zhubei City (TW)

(72) Inventors: Yung-Chu CHEN, Zhubei City (TW); Chi-Sheng Lo, Zhubei City (TW); Mei-Wen Yen, Zhubei City (TW)

(73) Assignees: ANDROS PHARMACEUTICALS CO., LTD., Zhubei (TW); ANDROS PHARMACEUTICALS CO., LTD., Zhubei (TW)

(21) Appl. No.: 18/426,213

(22) PCT Filed: Sep. 9, 2021
(86) PCT No.: PCT/CN2021/117393

§ 371 Date: Jan. 29, 2024

(51) Int. Cl. A61K 31/245; A61K 9/00; A61K 31/167; A61K 47/02; A61K 47/10; A61K 47/20; A61K 47/24; A61K 47/38

(52) U.S. Cl. CPC A61K 31/245; A61K 9/0014; A61K 31/167; A61K 47/02; A61K 47/10; A61K 47/20; A61K 47/24; A61K 47/38

(57) ABSTRACT

The present invention relates to a topical anesthetic containing anesthetic agent-clay composite as the anesthetic agent in a delivery vehicle suitable for administration to the skin for increasing anesthetic agent permeation into the skin in order to decreasing onset time. In addition, the compositions of the invention have been shown to exhibit good storage stability with low ester type of anesthetic agent decomposition even at relatively high storage temperatures.

### TECHNICAL FIELD

The present invention relates to the field of biomedical or biopharmaceutical technology and particularly to compositions and methods for the topical administration of pharmaceutically active agents to a mammal in need thereof. More particularly, the present invention relates to a topical anesthetic containing anesthetic agent-clay composite as the anesthetic agent in a delivery vehicle suitable.

### BACKGROUND OF THE INVENTION

With the emergence of surgical techniques, the need for more effective topical anesthesia continues to develop. Patients undergoing venepuncture or intravenous catheterization often experience pain. Therefore, a fast acting and long lasting topical anesthetic formulation would be of considerable clinical benefit in reducing pain associated with invasive medical procedures.

Anesthetic agents, such as lidocaine, prilocaine, mepivacaine and bupivacaine, have been shown to possess local anesthetic effects and are used for infiltration anesthesia and for inducing nerve blocks. These drugs have limited use as local anesthetics since they have to be given in high concentrations, which increase the risk of toxicity, skin irritation and skin damage.

Ester local anesthetics are also potent vasodilating drugs such as tetracaine. A significant clinical effect of vasodilation is an increase in the rate of absorption of the local anesthetic into the blood, thus decreasing the duration and quality of pain control, while increasing the anesthetic blood concentration and its potential for overdose. Hence, increasing anesthetics accumulation in local tissues is an important role in local anesthetic formulation design. In addition, local anesthetic esters or ester-type local anesthetics have been known to be susceptible to chemical degradation which can result in reduced concentrations of the local anesthetic and/or increase in certain impurities. Tetracaine is a particularly difficult local anesthetic to stabilize long term, especially in aqueous formulations. Tetracaine is sparingly soluble in water, dispersions in water will have pH values typically from pH 8 to 11 and under these pH conditions have a high rate of hydrolysis. In addition, tetracaine is unstable in the human body where practically all tissues contain esterases enzyme.

A number of references disclose local anesthetic compositions. For instance, U.S. Pat. No. 8,968,710B1 disclosed the vehicle for transporting the tetracaine includes a water soluble mucoadhesive such as a high molecular weight poly (ethylene oxide) homopolymer and a cellulose polymer. The vehicle addition ally includes propylene glycol which functions as penetration enhancer and is essential to (1) delivery of tetracaine free base in a stable form and (2) delivery at a pH which favors drug flux across the mucous membranes. The tetracaine is ground into a powder and is suspended in a plasticized hydrocarbon gel which completes the vehicle.

U.S. Pat. No. 6,562,326B1 disclosed a method of treating skin includes applying a topical composition to an affected area of skin, such as a burn, irritation, blister, rash or other similar skin condition. The topical composition has as the active ingredients an anesthetic and a surfactant. The anesthetic is preferably tetracaine in a concentration of from about 1% to 2% by weight and the surfactant is preferably sodium lauryl sulfate in a concentration of from about 0.5% to about 5.0% by weight.

US20070232695A1 disclosed a composition has a high concentration of tetracaine can be absorbed on a high surface area material, such as an ion exchange polymer and dispersed in the liquid vehicle. The composition can anesthetize the gingivae for an extended period by cation exchange to release drug in mouth.

U.S. Pat. No. 8,513,304B2 described a topical formulation comprising (1) an active agent selected from at least one of lidocaine and tetracaine; (2) a first compound, and (3) a second compound, wherein the first compound and second compound are different, and each is selected from the group consisting ofN-lauroyl sarcosine, sodium octyl sulfate, methyl laurate, isopropyl myristate, oleic acid, glyceryl oleate and sodium lauryl sulfoacetate. The topical formulation can improve fluxes of therapeutically active agents.

US20140163105A1 disclosed a composition for pain relief including synergistically effective amounts of an amino benzoate local anesthetic such as 2% tetracaine, methylsulfonylmethane, and ethoxydiglycol. A method of treating pain, by applying the composition to skin in an area of pain and blocking nerve signals.

Liu (Liu et al., International Journal of Pharmaceutics 305(1-2): p31-36, 2005) disclosed anesthetic gels containing 4% tetracaine in carbomer vehicle with and without menthol were prepared. The tetracaine gel with menthol conferred significantly higher diffusion of tetracaine across full-thickness mouse skin than tetracaine gel without menthol.

US20140205589 A1 disclosed a composition can alleviate or even annihilate cutaneous reactions and improved stability of its components. The composition can include an emulsion with an oil phase and an aqueous phase, wherein the oil phase is a eutectic mixture of at least one anesthetic compound such as tetracaine and at least one adrenergic receptor agonist such as brimonidine.

U.S. Pat. No. 5,227,165A disclosed a local anesthetic lipospheres, that are solid, water-insoluble microparticles that have a layer of a phospholipid embedded on their surface. The core of the liposphere is a solid anesthetic such as tetracaine. Anesthetic lipospheres provide a controlled delivery of local anesthetics to achieve extended, effective relief from pain by slowly releasing the anesthetic from the solid hydrophobic core.

U.S. Pat. No. 4,937,078A disclosed liposome encapsulated analgesic agents such as tetracaine when applied to skin or mucous membranes provided greater local anesthesia and analgesia than the same agents incorporated in conventional vehicles.

EP0522122B1 disclosed the presence of salt were shown to be the critical factors for reducing the rate of decomposition of tetracaine.

U.S. Pat. No. 3,272,700 indicated the presence of tyloxapol can inhibit the rate of decomposition of tetracaine.

U.S. Pat. No. 8,907,153B2 related generally to systems developed for dermal delivery of drugs. More particularly, the present invention relates to adhesive peel-forming formulations having a viscosity suitable for application to a skin surface, and which form a sustained drug-delivering adhesive solidified peelable layer on the skin.

U.S. Pat. No. 9,358,219B2 related to a topical formulation of multiplexed molecular penetration enhancers. In a particularly preferred embodiment, the present invention relates to a topical formulation of multiplexed molecular penetration enhancers for topical or transdermal administration of one or more active ingredients selected from lidocaine and tetracaine.

U.S. Pat. No. 6,325,990 disclosesd a composition for spraying on the skin and comprising a lipophilic active compound, including analgesics such as lidocaine, from 0.5-25% by weight of a silicone-based adhesive polymer composition; from 0-25% by weight of an absorption promoter; from 25-95% by weight of a volatile solvent comprising volatile silicones; and from 0.5-50% by weight of a pressurised propellant gas. Preferably, the volatile silicones represent from 50-85% by weight of the composition. Additionally, the compositions include up to 25% of a volatile solvent such as ethanol.

US20060147383 disclosed a pharmaceutically-active agent such as lidocaine hydrochloride contained in an alcoholic vehicle including at least one volatile silicone and a non-volatile oily phase, for administration by spraying. The volatile silicone is present at between 25 and 95% by weight of the composition. Preferably, an alcohol is also present as a solvent, at a concentration of at least 15%, preferably at least 25% of ethanol.

U.S. Pat. No. 9,308,181 disclosure is drawn to topical formulations, transdermal systems, and related methods. In one embodiment, a topical formulation is provided that includes a local anesthetic, a first compound, and a second compound. The first compound and second compound are different and each is selected from the group consisting of N-lauroyl sarcosine, sodium octyl sulfate, methyl laurate, isopropyl myristate, oleic acid, glyceryl oleate, and sodium lauryl sulfoacetate.

U.S. Pat. No. 6,528,086B2 related to an apparatus and method of drug delivery on a human body surface. The formulation comprises a drug, a conversion agent capable of converting the formulation from a less solid phase to a coherent, soft, solid phase, and a vehicle medium or carrier for the drug and conversion agent. The drug formulation is applied to this human body surface in its less than solid phase and is subsequently converted to a soft solid phase while the drug is being delivered through the human body surface. After delivery of the drug is complete, the soft solid formulation can be removed or peeled from the body surface as a coherent solid formulation.

U.S. Pat. No. 9,693,976 disclosed a solid-forming local anesthetic formulation for pain control can include a lidocaine base and tetracaine base, polyvinyl alcohol, water, and an emulsifier. The formulation can be prepared to be in a semi-solid state prior to application to a skin surface, can form a soft solidified layer after application, and can provide pain relief when applied to a skin surface proximate a pain site.

US20140205589A1 described a composition can reduced degradation rate and/or improved stability of its components. The composition can alleviate or even annihilate cutaneous reactions and can include an emulsion with an oil phase and an aqueous phase, wherein the oil phase can be a eutectic mixture of at least one anesthetic compound and at least one adrenergic receptor agonist. Methods of using such a composition are also described.

Although many local anesthetic compositions have been proposed, it has been discovered that the incorporation of one or more anesthetic agents in a solvent for the anesthetic agent into a pharmaceutically acceptable carrier, permitting more rapid delivery of the anesthetic agent to the tissue with aqueous formulations which may result in reduced concentrations of the local anesthetic and/or increase in certain impurities.

Skin is a structurally complex, relatively thick membrane. Molecules moving from the environment into and through intact skin must first penetrate the stratum corneum and any material on its surface. They must then penetrate the viable epidermis, the papillary dermis, and the capillary walls into the blood stream or lymph channels to be so absorbed; molecules must overcome a different resistance to penetration in each type of tissue. The skin is a multilayered structure that is designed to protect the body against undesired influences from the external environment. The main barrier is located in the upper layer of the skin, the stratum corneum, is the major problem for transdermal and dermal delivery of drugs. This impermeability may be attributed to the nature of one very thin layer created by normal development and physiological changes in the skin. After cells are formed in the basal layer, they begin to migrate toward the skin surface, until they are eventually sloughed off. As they undergo this migration, they become progressively more dehydrated and keratinized. When they reach the surface, just prior to being discarded, they form a thin layer of dense, metabolically inactive cells approximately ten microns thick. As a result of the high degree of keratinization of the cells, which comprise the stratum corneum, a formidable barrier is created. Absorption through a mucosal surface is generally efficient because the stratum corneum is absent. Therefore, any formulation to be utilized as an efficient topical, transdermal anesthetic must be capable of being readily absorbed through the skin.

Skin occlusion is an effective means of absorbing ointments and medication because it leaves your skin hydrated. If you leave a patch or plastic film dressing on your skin, the moisture from your body has nowhere to go, trapping in extra hydration. This excess hydration makes it easier for the active ingredient or medicines to permeate into skin, such as Ametop gel must be covered with a plastic film dressing for 30-45 minutes for it work. Another way to obtain penetration of the active ingredient or medicines into the skin is to provide occlusion by formulating the active ingredient in a hydrophobic vehicle such as petrolatum. However, ointments containing petrolatum generally have a tacky or greasy feel that persists for quite some time after application. As an alternative to conventional formulations such as ointments, compositions containing film-forming polymers in which an active ingredient has been incorporated have been developed. Film-forming compositions have mainly been used to provide transdermal delivery of an active ingredient such as in transdermal patches or, more recently, as film-forming solutions composed of a film-forming polymer, a plasticiser and a low-molecular volatile solvent for the active ingredient. When the solution is applied on skin, a thin polymeric film is formed after evaporation of the solvent. For example, U.S. Pat. No. 8,907,153B2 disclosed an adhesive peel-forming formulation for dermal delivery of a drug, comprising a drug, a volatile solvent system, a non-volatile solvent system and a peel-forming agent. The peel-forming agent is selected from the group consisting of polyvinyl alcohol, polyvinyl pyrrolidone, carrageenan, gelatin, dextrin, guar gum, xanthan gum, polyethylene oxide having a weight average molecular weight greater than about 5,000 Mw, starch, cellulose derivatives. However, the film-forming formulations need to wait evaporation of the solvent so that the film-forming formulations achieving onset time is longer than the formulations be covered with a plastic film dressing.

A topical eutectic mixture local anesthetic EMLA cream (a cream containing 2.5% lidocaine and 2.5% prilocaine) is available clinically. However, it requires a minimum anesthesia onset time and duration. Tetracaine free base is a surface anesthetic agent and more lipophilicity. It penetrates through stratum corneum more easily than the EMLA cream (Romsing et al., 1999). Tetracaine free base is a local anesthetic of the ester type with formula which contains 40 mg of active ingredient (4%, w/w), an aqueous gelling agent and a pharmaceutically acceptable salt. AMETOP would offer an improved local anesthesia but what has been lacking is a shelf stable formulation capable of delivering the tetracaine free base in delivery system. A recently introduced topical anaesthetic, which contains the highest concentration of active anaesthetic ingredients available in a TFDA and US FDA approved topical cream, is an eutectic mixture of 7% lidocaine and 7% tetracaine cream (Pliaglis-Galderma S.A.). As Pliaglis cream forms a self-occlusive film when exposed to air, it is said to not require plastic occlusion. Pliaglis should be applied onto intact skin at a thickness of approximately 1 mm for 30 minutes or 60 minutes (approximately 1.3 g of cream per 10 cm²) in different dermatological procedures. Pliaglis has the same storage stability problem as AMETOP, and it must be stored at 2 to 8° C.

Clay in the soil is microscopic and can be an arrangement of only a few atoms. Because clay is so small, large amounts of clay together create lots of tiny in-between spaces. Since larger soil particles such as sand are solid, the total volume of space between them is less than the volume of the space between an equal amount of clay particles. The increased amount of spaces between clay particles creates abundant surface area on which water molecules can adhere. Clay is so fine and has such a high surface area per unit of volume it can absorb huge amounts of water and has a higher water-holding capacity. Thus, clay maybe decrease the degree of decomposition of tetracaine by water-adsorbing ability of clay. In addition, clay maybe also increase skin penetration of the active ingredient by forming occlusive effect of clay and water-holding capacity of clay to increase hydration effect.

Summing up, there is a need in the art for a novel anesthetic composition. Hence, efficient drug permeation into the skin to decrease onset time and good shelf stable is essential to an ideal topical anesthetic formulation.

### SUMMARY OF THE INVENTION

The object of the invention is to provide an anesthetic agent-clay composite composition in which the tetracaine will not significantly degrade but remains chemically stable throughout the shelf-life of the composition. Another object of the invention is to develop an anesthetic agent-clay composite composition of self-occlusive effect that can help anesthetic agent permeate quickly into the skin so that the onset time of the composite composition is shorter than film-forming compositions.

In the first aspect, it provides an anesthetic composition for topical application comprising:
(a) therapeutically effective amount of at least one pharmaceutically active agent;
(b) at least one pharmaceutically acceptable solvent vehicle;
(c) at least one clay;
the pharmaceutically active agent is ester type or amide type anesthetic agent.

Preferably, the pharmaceutically active agent is substantially dissolved in the solvent so that when mixed with the clay or carrier, the agent is microdispersed in the composition.

In preferred embodiments, the composition is in semi-solid form and store at ambient temperatures.

In preferred embodiments, the ester type anesthetic agent is in an amount of about 1 to 10% based on the weight of the whole composition.

In preferred embodiments, the amide type anesthetic agent is in an amount of about 1 to 10% based on the weight of the whole composition.

In particular preferred embodiments, the ester type anesthetic agent is in an amount of 5% or 7% based on the weight of the whole composition.

In particular preferred embodiments, the amide type anesthetic agent is in an amount of 5% or 7% based on the weight of the whole composition.

In preferred embodiments, the clay represents about 5 to 60% based on the weight of the whole composition.

In particular preferred embodiments, the clay represents 13 to 56% based on the weight of the whole composition.

In particular preferred embodiments, the anesthetic agent is selected from the group consisting of benzocaine, chloroprocaine, cyclomethycaine, dimethocaine, piperocaine, propoxycaine, procaine, proparacaine, tetracaine, lidocaine, mepivacaine, prilocaine, bupivacaine, ropivacaine and articaine.

In another particular preferred embodiments, the clay is selected from the group consisting of nanosilicate platelets, montmorillonite, bentonite, mica, laponite, kaolin, talc, hydrotalcite, attapulgite clay, vermiculite, hectorite, saponite, stevensite, beidellite or layered double hydroxides.

In preferred embodiments, the said anesthetic composition comprise at least one excipient.

In preferred embodiments, the said anesthetic composition comprise a pharmaceutically acceptable solvent vehicle, wherein the carrier is selected from the group consisting of methanol, ethanol, propyl glycol, ethyl acetate, isopropyl alcohol, transcutol P, PEG300, PEG400, glycerol, acetone, lauryl alcohol, oleyl alcohol, cyclomethicone, methylene chloride, benzyl alcohol, acetone, acetic acid, propylene carbonate, dichloromethane, chloroform, 1,4-dioxane, dimethylformamide, dimethyl sulphoxide, toluene, tetrahydrofuran, dodecanol, water or their combination.

In particular preferred embodiments, the said anesthetic composition comprises:
(a) 1 to 10 weight percent of anesthetic agent; and
(b) 5 to 60 weight percent of talc; and
(c) 20 to 90 weight percent of pharmaceutically acceptable vehicle; and
(d) 1 to 5 weight percent of HPC.

In particular preferred embodiments, the said anesthetic composition comprises 5 weight percent of tetracaine and 56 weight percent of talc.

In particular preferred embodiments, the said anesthetic composition comprises 7 weight percent of tetracaine, 7 weight percent of lidocaine, and 24 weight percent of talc.

In particular preferred embodiments, the said anesthetic composition comprises 5 weight percent of tetracaine, 56 weight percent of talc, and 35 weight percent of DMSO (Dimethyl sulfoxide), 1 weight percent of HPC (hydroxypropyl cellulose).

In particular preferred embodiments, the said anesthetic composition comprises 7 weight percent of tetracaine, 7 weight percent of lidocaine, 24 weight percent of talc, 32 weight percent of dicalcium phosphate, 20 weight percent of DMSO, 3 weight percent of transcutol p, and 1 weight percent of HPC.

In particular preferred embodiments, the said anesthetic composition comprises 7 weight percent of tetracaine, 7 weight percent of lidocaine, 24 weight percent of talc, 32 weight percent of dicalcium phosphate, 23 weight percent of DMSO, 6 weight percent of petrolatum, and 1 weight percent of HPC.

In the second aspect, the present invention provides a method of administering one or more pharmaceutically active agents to a subject comprising the steps of:
(a) providing the said anesthetic composition; and
(b) contacting an area of skin with the anesthetic composition.

### BRIEF DESCRIPTION OF THE DRAWINGS

FIG. 1 show the results of efficacy study of tetracaine-clay composite formulation and tetracaine-PEG formulation. It depicts that the skin permeation of the tetracaine-clay composite formulation was faster than the tetracaine-PEG formulation.

FIG. 2 show the results of the duration of the anesthetic effect. It depicts that the duration of the tetracaine-clay composite formulation was longer than the tetracaine-PEG formulation.

FIG. 3 show the results of efficacy study of tetracaine-clay composite formulation M and commercial product (AMETOP). It depicts that the penetration amount of the tetracaine-clay composite formulation M was higher than the AMETOP.

FIG. 4 show the results of efficacy study of tetracaine-clay composite formulation M and commercial product (AMETOP). It depicts that the skin permeation of the tetracaine-clay composite formulation M was faster than the AMETOP.

FIG. 5 show the results of the duration of the anesthetic effect. It depicts that the duration of the tetracaine-clay composite formulation M was longer than the commercial product (AMETOP).

FIG. 6 show the results of efficacy study of tetracaine/lidocaine-clay composite formulation N and commercial product (Pliaglis). It depicts that the tetracaine-clay composite formula N entered the skin faster than Pliaglis.

FIG. 7 show the results of efficacy study of tetracaine/lidocaine-clay composite formulation O and commercial product (Pliaglis). It depicts that the tetracaine-clay composite formula O entered the skin faster than Pliaglis.

FIG. 8 show the results of efficacy study of tetracaine-clay composite formulation P and commercial product (EMLA). It depicts that the penetration amount of the tetracaine-clay composite formulation P was higher than the EMLA.

FIG. 9 show the results of efficacy study of tetracaine-clay composite formulation Q and commercial product (EMLA). It depicts that the penetration amount of the tetracaine-clay composite formulation Q was higher than the EMLA.

### DETAILED DESCRIPTION OF THE INVENTION

Through extensive and intensive research and extensive screening, the inventors unexpectedly obtained a topical anesthetic agent composition which is provided for administration to the skin to decrease onset time. The topical anesthetic agent composition may be used to numb the skin before a medical procedure. As compared to conventional topical anesthetics, the subject anesthetic agent composition has demonstrated increased anesthetic agent permeation into the skin and improved shelf life.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a”, “an”, and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises”, “comprising”, “includes”, “including”, “has”, “having”, “contains” or “containing”, or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of”, or variations such as “consist of” or “consisting of”, as used throughout the specification and claims, indicate the inclusion of any recited integer or group of integers, but that no additional integer or group of integers can be added to the specified method, structure, or composition.

As used herein, the term “consists essentially of”, or variations such as “consist essentially of” or “consisting essentially of”, as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about”, “approximately”, “generally”, “substantially”, and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The term “microdispersed” is intended to mean that in the solvent, and subsequently the carrier, there is an intimate dispersion of the pharmaceutically active agent at the molecular or ionic level, such that crystals of the pharmaceutically active agent cannot be detected using a microscope having a magnification of 25 times.

The term “therapeutically effective amount” is intended to mean the amount of drug sufficient to produce an anesthetic effect when applied topically. These amounts are known in the art or may be determined by methods known in the art, and typically range from about 1 to 20,000 mg per human adult and preferably about 10 to 10,000 mg and most preferably range from about 20 to 5,000 mg of the anesthetic agent per application, depending upon the anesthetic agents chosen, and whether the tissue, such as the skin or mucous membrane is the site of action. The only upper limit on the amount of anesthetic in the composition is that the preparation is substantially free of crystals of anesthetic agent and the amount of solvent used is not sufficient to undesirably affect the adhesive properties of the finite composition. Thus, the single ingredient anesthetic agent contains a therapeutically effective amount of anesthetic agent within the foregoing range.

The concentration as well as the quantity of anesthetic per unit area, namely per square or cubic centimeter can be varied independently in order to achieve the desired effect. Higher concentrations of anesthetic base contained in a dosage form of decreased thickness will result in an anesthetic with fast onset and short duration. High concentrations of the anesthetic base contained in a dosage form of increased thickness (higher mg of anesthetic per square or cubic centimeter) will result in potent anesthesia with fast onset and long duration. Low concentrations of the anesthetic base in a dosage form of decreased thickness will result in mild anesthesia with longer onset and short duration. Low concentrations of the anesthetic base contained in a dosage form of increased thickness will have mild anesthesia with longer onset and longer duration. As shown in the above explanation, the ability to vary the concentration of anesthetic from very low (about 1%) to high (40% or higher) of the total composition, when combined with the ability to coat thin or thick enables the practitioner of the invention to vary the dosage of the system as needed for particular anatomical sites of interest.

As a general rule, in the case of a given tissue the anesthetic drug selected, the concentration and thickness and the duration of the application is determined based upon the anesthetic's ability to penetrate the tissue and to be at peak effectiveness within about 2 to 60 minutes. The duration of the effect of the anesthetic on the tissue, for example the oral mucosa, should range between about 2 to 240 minutes, depending on the anesthetic agent selected, the concentration of the anesthetic and the thickness of application. Longer or shorter durations can also be selected dependent on need, as will be apparent to one skilled in the art.

The term “onset of anesthesia” is intended to mean the time to peak effect on the individual nerves. Onset of anesthesia principally depends upon the lipid solubility, molecular size, and quantity of available, un-ionized form of the local anesthetic. Thus, anesthetics with a high lipid solubility or a low pKa, or both, have a more rapid onset of anesthesia.

The term “duration of anesthesia” as used herein means the period of time during which the local anesthetic measurably blocks nerve conduction. The foregoing depends upon all of the factors listed for onset of anesthesia, as well as on the extent of protein binding of the anesthetic agent.

The anesthetic agent-clay composite compositions of the invention comprise one or more anesthetic agent, one or more clay, one or more solvent vehicle. The solvent vehicle can comprise one or more solvents, wherein the solvent system provides a window of operable solubility for the anesthetic agent. The clay provides a higher water-adsorbing ability to decrease the degree of decomposition of anesthetic agent and also form occlusive effect and provide water-holding capacity to increase hydration effect of skin.

The addition of clay has been found to improve chemically stable of the anesthetic and without reducing the rate of drug delivery. Suitable clays include talc, hectorite, attapulgite clay, nanosilicate platelets, laponite, mica, vermiculite, saponite, stevensite, beidellite, kaolin such as boalinite, anauxite, dickite and nacrite, montmorillonites such as montmorillonite, bentonite, bordellite and montronite, illites/muscovites such as illite and glauconite, chlorites, polygorshites such as attapulgite, halloysite, metabolloysite, allophane and aluminum silicate clays. Especially useful are smectite clays. For most formulations, the weight percentage of the clays can be from about 5 wt % to about 60 wt %.

The anesthetic agents of this invention are those known, or of a type known, in the art. Local anesthetic agents suitable for use in the practice of this invention include amides and esters. Examples of the amides are lidocaine, prilocaine, mepivacaine, bupivacaine, dibucaine and etidocaine. Esters include procaine, tetracaine, propoxycaine, chloroprocaine, benzocaine, butamben picrate, cocaine, hexylcaine, piperocaine, oxyprocaine and proparacaine. Other suitable local anesthetics for use in the practice of this invention include cyclomethycaine, dimethisoquin, ketocaine, diperodon, dyclonine and pramoxine, all typically administered in the form of the acid addition hydrochloride or sulfate salts.

The amount of anesthetic agent in the formulation will vary depending on the desired therapeutic effect and duration of anesthesia needed. In the present invention, the concentration of the anesthetic agent is 1 to 10 wt % of the total composition to deliver an effective dosage.

Local anesthetics can be used topically or injected. The anesthetic agent-clay composite compositions can contain one or more pharmaceutically acceptable excipients. Suitable excipients include, but are not limited to, diluents, dispersing agents, solubilizing agents, surfactants, stabilizing agents, pH adjusting agents, colorants, preservatives, and humectants.

In addition to the above ingredients, there may also be incorporated other additives selected from among the various pharmaceutically acceptable additives available to those skilled in the art. These additives include binders, preservatives, flavorings and pigments.

In some embodiment, the resulting mixture is in semi-solid form such as a cream, gel, emulsion, lotion, salve, plaster, paste, ointment, spray-solution or other “non-finite” composition. The final form in which the composition of the invention will be applied depends upon the anatomical site of application and ease of access.

The solvent vehicle can be selected from pharmaceutically or cosmetically acceptable solvents. Examples of solvents include methanol, ethanol, propyl glycol, ethyl acetate, isopropyl alcohol, transcutol P, PEG300, PEG400, glycerol, acetone, lauryl alcohol, oleyl alcohol, cyclomethicone, methylene chloride, benzyl alcohol, acetone, acetic acid, propylene carbonate, chloroform, 1,4-dioxane, dimethylformamide, dimethyl sulphoxide, toluene, tetrahydrofuran, dodecanol, water or the like. Additionally, these solvents should be chosen to be compatible with the rest of the formulation and the compositions can contain one or more solvents. For most formulations, the weight percentage of the solvents can be from about 5 wt % to about 50 wt %.

The composition of this invention can be manufactured by numerous methods known in the art which permit the achievement of a microdispersed anesthetic agent, including extruding, molding, solvent casting, coating, and all other methods which employ a solvent to disperse the drug in a finite or non-finite carrier. The anesthetic agent-clay composite compositions can be in virtually any form, for example, lotions, ointments, creams, gels, drops, suppositories, sprays, liquids, solutions, and powders.

In some embodiment, the term “pharmaceutically acceptable carrier” is intended to be any suitable finite or non-finite carrier including liquids, semi-liquids or solid carriers, such as a bioadhesive. Thus, the active agents may be admixed with a non-adhesive tape or other finite carrier or a carrier, or any other “non-finite” carrier known in the art of pharmaceutical delivery. For example, the base of a non-finite carrier may be fatty oils, lanolin, vaseline, paraffins, glycols, higher fatty acids and higher alcohols.

The main advantages of the present invention include:
(a) exhibiting good storage stability with low ester type of anesthetic agent decomposition even at relatively high storage temperatures;
(b) increasing anesthetic agent permeation into the skin in order to decreasing onset time;
(c) increasing the duration of the anesthetic effect.

The present invention is further described by reference to the following examples. It should be understood that the following examples are only used to describe the present invention, rather than limiting the scope of the present invention. The experimental methods in the following examples, the specific conditions of which are not indicated, are usually carried out according to conventional conditions, or the conditions recommended by the manufacturers. Unless otherwise specified, percentages and parts refer to percentages by weight and parts by weight.

### EXAMPLE 1. CHEMICAL STABILITY FOR TETRACAINE-TALC COMPOSITE FORMULATION

Table 1 shows the effect of different talc concentrations on the inhibition of tetracaine impurities. Generally, all formulations were manufactured as described below. The liquid phase was prepared by mixing the mixture of the tetracaine, dimethyl sulfoxide (DMSO), cyclomethicone and water at room temperature. Subsequently, the talc was added at the same temperature to form tetracaine-talc composite formulation. The final product was then tested for the chemical properties and placed on stability at appropriate storage conditions. The details of the formulation and the result of the stability experiments are set out in Table 1.

Each of the formulations described in Example 1 were tested for the chemical stability of tetracaine following storage at 40° C. The chemical stability was measured in terms of the generation of the impurity of tetracaine. Specifically, tetracaine is known to break down, so a lower concentration of this impurity after a period of weeks demonstrates greater tetracaine stability over time. Every formulation in accelerated conditions was tested at 1 month.

The chemical stability of the formulations was assessed by measuring the concentration of the tetracaine and formation of impurities using High Performance Liquid Chromatographic (HPLC) method. The HPLC method involves chromatographic separation by mobile phase gradient and C18 analytical column; and quantification of each component by ultraviolet (UV) detector. Results of stability under accelerated temperature condition are provided in Table 1. These results further demonstrate the effect of inclusion of talc with Formulations A containing 48% talc showing only 0.06% decomposition of tetracaine at 40° C. compared to about 1.86% and 5.54% decomposition in Formulations B and C containing 28% and 13% talc, respectively. The data on rate of decomposition at 40° C. show that the degradation products of tetracaine will decrease when the concentration of talc increases.

TABLE 1

| | | | | | | Impurity |
|---|---|---|---|---|---|---|
| | Composition (wt %) | | | | | (%) |
| Formu- | Tetracaine | | | cyclo- | | 40° C., |
| lation | (TTC) | talc | DMSO | methicone | water | 1 month |
| A | 5 | 48 | 35 | 5 | 7 | 0.06 |
| B | 5 | 28 | 35 | 5 | 27 | 1.86 |
| C | 5 | 13 | 35 | 5 | 42 | 5.54 |

### EXAMPLE 2. CHEMICAL STABILITY FOR TETRACAINE-BENTONITE COMPOSITE FORMULATION

Table 2 shows the effect of different bentonite concentrations on the inhibition of tetracaine impurities. Generally, all formulations were manufactured as described below. The liquid phase was prepared by mixing the mixture of the tetracaine, dimethyl sulfoxide (DMSO), cyclomethicone and water at room temperature. Subsequently, the bentonite was added at the same temperature to form tetracaine-bentonite composite formulation. The final product was then tested for the chemical properties and placed on stability at appropriate storage conditions. The details of the formulation and the result of the stability experiments are set out in Table 2.

Each of the formulations described in Example 2 were tested for the chemical stability of tetracaine following storage at 40° C. The chemical stability was measured in terms of the generation of the impurity of tetracaine. Specifically, tetracaine is known to break down, so a lower concentration of this impurity after a period of weeks demonstrates greater tetracaine stability over time. Every formulation in accelerated conditions was tested at 1 month.

The chemical stability of the formulations was assessed by measuring the concentration of the tetracaine and formation of impurities using High Performance Liquid Chromatographic (HPLC) method. The HPLC method involves chromatographic separation by mobile phase gradient and C18 analytical column; and quantification of each component by ultraviolet (UV) detector. Results of stability under accelerated temperature condition are provided in Table 2. These results further demonstrate the effect of inclusion of bentonite with Formulations D containing 13% bentonite showing 9.97% decomposition of tetracaine at 40° C. compared to about 11.48% decomposition in Formulations E containing 8% bentonite. The data on rate of decomposition at 40° C. show that the degradation products of tetracaine will decrease when the concentration of bentonite increases.

TABLE 2

| | | | | | | Impurity |
|---|---|---|---|---|---|---|
| | Composition (wt %) | | | | | (%) |
| Formu- | Tetracaine | | | cyclo- | | 40° C., |
| lation | (TTC) | bentonite | DMSO | methicone | water | 1 month |
| D | 5 | 13 | 35 | 5 | 42 | 9.97 |
| E | 5 | 8 | 35 | 5 | 47 | 11.48 |

### EXAMPLE 3. CHEMICAL STABILITY FOR TETRACAINE-KAOLIN COMPOSITE FORMULATION

Table 3 shows the effect of different kaolin concentrations on the inhibition of tetracaine impurities. Generally, all formulations were manufactured as described below. The liquid phase was prepared by mixing the mixture of the tetracaine, dimethyl sulfoxide (DMSO), cyclomethicone and water at room temperature. Subsequently, the kaolin was added at the same temperature to form tetracaine-kaolin composite formulation. The final product was then tested for the chemical properties and placed on stability at appropriate storage conditions. The details of the formulation and the result of the stability experiments are set out in Table 3.

Each of the formulations described in Example 3 were tested for the chemical stability of tetracaine following storage at 40° C. The chemical stability was measured in terms of the generation of the impurity of tetracaine. Specifically, tetracaine is known to break down, so a lower concentration of this impurity after a period of weeks demonstrates greater tetracaine stability over time. Every formulation in accelerated conditions was tested at 1 month.

The chemical stability of the formulations was assessed by measuring the concentration of the tetracaine and formation of impurities using High Performance Liquid Chromatographic (HPLC) method. The HPLC method involves chromatographic separation by mobile phase gradient and C18 analytical column; and quantification of each component by ultraviolet (UV) detector. Results of stability under accelerated temperature condition are provided in Table 3. These results further demonstrate the effect of inclusion of talc with Formulations F containing 28% kaolin showing only 3.19% decomposition of tetracaine at 40° C. compared to about 6.10% and 7.77% decomposition in Formulations G and H containing 13% and 8% kaolin, respectively. The data on rate of decomposition at 40° C. show that the degradation products of tetracaine will decrease when the concentration of kaolin increases.

TABLE 3

| | | | | | | Impurity |
|---|---|---|---|---|---|---|
| | Composition (wt %) | | | | | (%) |
| Formu- | Tetracaine | | | cyclo- | | 40° C., |
| lation | (TTC) | kaolin | DMSO | methicone | water | 1 month |
| F | 5 | 28 | 35 | 5 | 27 | 3.19 |
| G | 5 | 13 | 35 | 5 | 42 | 6.10 |
| H | 5 | 8 | 35 | 5 | 47 | 7.77 |

### EXAMPLE 4. CHEMICAL STABILITY FOR TETRACAINE-CLAY COMPOSITE FORMULATION

Table 4 shows the effect of type of clay on the inhibition of tetracaine impurities. Generally, all formulations were manufactured as described below. The liquid phase was prepared by mixing the mixture of the tetracaine, dimethyl sulfoxide (DMSO), cyclomethicone and water at room temperature. Subsequently, the bentonite, talc or kaolin was added at the same temperature to form tetracaine-clay composite formulation. The final product was then tested for the chemical properties and placed on stability at appropriate storage conditions. The details of the formulation and the result of the stability experiments are set out in Table 4.

Each of the formulations described in Example 4 were tested for the chemical stability of tetracaine following storage at 40° C. The chemical stability was measured in terms of the generation of the impurity of tetracaine. Specifically, tetracaine is known to break down, so a lower concentration of this impurity after a period of weeks demonstrates greater tetracaine stability over time. Every formulation in accelerated conditions was tested at 1 month.

The chemical stability of the formulations was assessed by measuring the concentration of the tetracaine and formation of impurities using High Performance Liquid Chromatographic (HPLC) method. The HPLC method involves chromatographic separation by mobile phase gradient and C18 analytical column; and quantification of each component by ultraviolet (UV) detector. Results of stability under accelerated temperature condition are provided in Table 4. These results further demonstrate the effect of inclusion of bentonite with Formulations D containing 13% bentonite showing 9.97% decomposition of tetracaine at 40° C. These results demonstrate the effect of inclusion of talc with Formulations C containing 13% talc showing 5.54% decomposition of tetracaine at 40° C. These results also demonstrate the effect of inclusion of bentonite with Formulations G containing 13% kaolin showing 6.10% decomposition of tetracaine at 40° C. The data on rate of decomposition at 40° C. show that the degradation products of tetracaine-talc formulation was less than tetracaine-bentonite formulation and tetracaine-kaolin formulation.

TABLE 4

| | | | | | | | | Impurity |
|---|---|---|---|---|---|---|---|---|
| | Composition (wt %) | | | | | | | (%) |
| | Tetracaine | | | | | | | 40 C., |
| Formulation | (TTC) | bentonite | talc | kaolin | DMSO | cyclomethicone | water | 1 month |
| D | 5 | 13 | 0 | 0 | 35 | 5 | 42 | 9.97 |
| C | 5 | 0 | 13 | 0 | 35 | 5 | 42 | 5.54 |
| G | 5 | 0 | 0 | 13 | 35 | 5 | 42 | 6.10 |

### EXAMPLE 5. CHEMICAL STABILITY FOR TETRACAINE-CLAY COMPOSITE FORMULATION AND TETRACAINE-PEG FORMULATION

The experiment is to evaluate the stability of talc and polymer to tetracaine (TTC). Table 5 shows the effect of type of clay on the inhibition of tetracaine impurities. Generally, all formulations were manufactured as described below. The base composition used for Formulation I of a carrier composition comprising tetracaine, isostearyl alcohol (ISAL), ethanol, talc, dimethyl sulfoxide (DMSO) and water. The liquid phase was prepared by mixing the mixture of the tetracaine, dimethyl sulfoxide (DMSO), isostearyl alcohol (ISAL), ethanol, and water at room temperature (RT). Subsequently, the talc was added at the same temperature to form tetracaine-clay composite formulation. The base composition used for Formulation J of a carrier composition comprising tetracaine, isostearyl alcohol (ISAL), ethanol, polyethylene glycol 6000 (PEG6000), dimethyl sulfoxide and water. The formulation was prepared by mixing the mixture of the tetracaine, dimethyl sulfoxide (DMSO), isostearyl alcohol (ISAL), ethanol, polyethylene glycol 6000 (PEG6000), and water at 50° C. Then, place the mixture at room temperature to form ointment. The final product was then tested for the chemical properties and placed on stability at appropriate storage conditions. The details of the formulation and the result of the stability experiments are set out in Table 5.

Each of the formulations described in Example 5 were tested for the chemical stability of tetracaine following storage at room temperature. The chemical stability was measured in terms of the generation of the impurity of tetracaine. Specifically, tetracaine is known to break down, so a lower concentration of this impurity after a period of weeks demonstrates greater tetracaine stability over time.

The chemical stability of the formulations was assessed by measuring the concentration of the tetracaine and formation of impurities using High Performance Liquid Chromatographic (HPLC) method. The HPLC method involves chromatographic separation by mobile phase gradient and C18 analytical column; and quantification of each component by ultraviolet (UV) detector. Results of stability under room temperature condition are provided in Table 5. These results further demonstrate the effect of inclusion of talc with Formulations I containing 28% talc showing 2.12% decomposition of tetracaine at room temperature. These results demonstrate the effect of inclusion of PEG6000 with Formulations J containing 28% PEG6000 showing 6.93% decomposition of tetracaine at room temperature. The data on rate of decomposition at room temperature show that the degradation products of tetracaine-talc formulation was less than tetracaine-PEG6000 formulation.

TABLE 5

| | | | | | | | | Impurity |
|---|---|---|---|---|---|---|---|---|
| | Composition (wt %) | | | | | | | (%) |
| | Tetracaine | | | | | | | RT, |
| Formulation | (TTC) | Talc | PEG6000 | DMSO | ISAL | Ethanol | water | 5 months |
| I | 5 | 28 | 0 | 35 | 3 | 7 | 22 | 2.12 |
| J | 5 | 0 | 28 | 35 | 3 | 7 | 22 | 6.93 |

### EXAMPLE 6. CHEMICAL STABILITY FOR BENZOCAINE-CLAY COMPOSITE FORMULATION AND BENZOCAINE-PEG FORMULATION

The experiment is to evaluate the stability of talc and polymer to benzocaine (BZC). Table 6 shows the effect of type of clay on the inhibition of benzocaine impurities. Generally, all formulations were manufactured as described below. The base composition used for Formulation R of a carrier composition comprising benzocaine, polyethylene glycol 4000 (PEG4000), dimethyl sulfoxide, sodium hydroxide and water. The formulation was prepared by mixing the mixture of the benzocaine, dimethyl sulfoxide (DMSO), polyethylene glycol 4000 (PEG4000), sodium hydroxide and water at 50° C. The base composition used for Formulation S of a carrier composition comprising benzocaine, talc, dimethyl sulfoxide (DMSO), sodium hydroxide and water. The liquid phase was prepared by mixing the mixture of the benzocaine, dimethyl sulfoxide (DMSO), sodium hydroxide and water at room temperature (RT). Subsequently, the talc was added at the same temperature to form benzocaine-clay composite formulation. The final product was then tested for the chemical properties and placed on stability at appropriate storage conditions. The details of the formulation and the result of the stability experiments are set out in Table 6.

Each of the formulations described in Example 6 were tested for the chemical stability of benzocaine following storage at 60° C. The chemical stability was measured in terms of the generation of the impurity of benzocaine. Specifically, benzocaine is known to break down, so a lower concentration of this impurity after a period of one week demonstrates greater benzocaine stability over time.

The chemical stability of the formulations was assessed by measuring the concentration of the benzocaine and formation of impurities using High Performance Liquid Chromatographic (HPLC) method. The HPLC method involves chromatographic separation by mobile phase gradient and C18 analytical column; and quantification of each component by ultraviolet (UV) detector. Results of stability under room temperature condition are provided in Table 6. These results demonstrate the effect of inclusion of PEG4000 with Formulations R containing 30% PEG4000 showing 6.6% decomposition of benzocaine at 60° C. These results further demonstrate the effect of inclusion of talc with Formulations S containing 30% talc showing 1.6% decomposition of benzocaine at 60° C. The data on rate of decomposition at room temperature show that the degradation products of benzocaine-talc formulation was less than benzocaine-PEG4000 formulation.

TABLE 6

| | Composition (wt %) | | | | | | Impurity (%) |
|---|---|---|---|---|---|---|---|
| | Benzocaine | | | | | | 60° C., |
| Formulation | (BZC) | Talc | PEG4000 | DMSO | water | NaOH | 4 days |
| R | 5 | 0 | 30 | 35 | 29.98 | 0.02 | 6.6 |
| S | 5 | 30 | 0 | 35 | 29.98 | 0.02 | 1.6 |

### EXAMPLE 7. EFFICACY STUDY FOR TETRACAINE-CLAY COMPOSITE FORMULATION AND TETRACAINE-PEG FORMULATION

The experiment is to evaluate the effect of talc and polymer on the efficacy of tetracaine (TTC). The Formulation K and Formulation L were applied to each Sprague Dawley (SD) rat (female rat, obtained from BioLASCO Taiwan Co., Ltd.) respectively. The application time was calculated from the time when the drug was applied. After 30 minutes of application, the drug in the test area was wiped off with gauze to perform von Frey test.

In this experiment, the mechanical sensitivity of the hind paw of rats was examined by Von Frey assay to evaluate the anesthetic effect.

The von Frey hair used in the experiment is a set of nylon needles with different thicknesses. Each von Frey hair has different strengths when puncture, from weak to strong in order of 4, 6, 8, 10, 15, 26, 60 g, a total of 7 nylon needles, which are physical stimuli during testing. Each rat was individually placed in a transparent feeding cage and placed on a stainless steel mesh (6.3×6.3 mm) for an adaptation time of 10 minutes, followed by a strength of von Frey hair. Vertical to the hind paw, the hind paw is pricked from the gap of the stainless steel mesh, and the von Frey hair is maintained in a curved state for 5 seconds, called a stimulus. Each intensity requires 5 stimuli at random locations on the hind paw. The von Frey hairs of different strengths are tested from weak to strong. After completing a strength test, the strength is replaced until the threshold is measured and recorded.

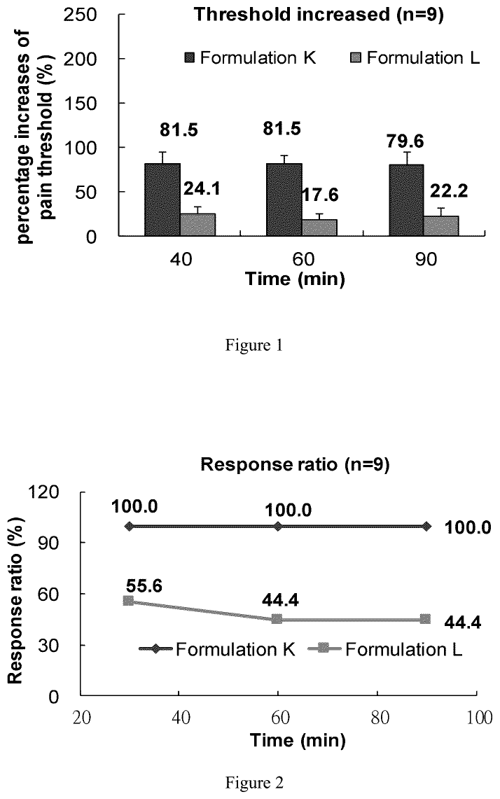

The details of each formulation and the results of the efficacy study are set out in Table 7, FIG. 1, and FIG. 2. In FIG. 1, it can be seen that Formulation K was more effective at increasing pain threshold when compared to Formulation L. The increased percentage of threshold is 3.4 times that of Formulation L at 40 min. The result showed that the drug of Formulation K entered the skin faster than Formulation L. In FIG. 2, the result also showed that 100% response ratio in the formulation K group still had anesthetic effect at 90 min, while 44.4% response ratio in the formulation L group still had anesthetic effect, indicated that the duration of Formulation K was longer than Formulation L.

TABLE 7

| | Composition (wt %) | | | | |
|---|---|---|---|---|---|
| Formulation | Tetracaine (TTC) | Talc | PEG4000 | DMSO | water |
| K | 5 | 56 | 0 | 30 | 9 |
| L | 5 | 0 | 56 | 30 | 9 |

### EXAMPLE 8. SKIN PERMEATION FOR TETRACAINE-CLAY COMPOSITE FORMULATION AND COMMERCIAL PRODUCT

Formulation M and the commercial topical tetracaine AMETOP were tested for permeation through porcine skin using the Franz diffusion cells with a 6 ml receptor well volume. The porcine skin was shaved free of hair, washed with phosphate buffered saline (PBS) and subcutaneous fat was removed. The donor well had an area of 0.785 cm². Receptor wells were filled with isotonic PBS doped. The flanges of the Franz cell were clamped together with uniform pressure using a pinch clamp. Receptor wells of the Franz cells were maintained at 32° C. in a stirring block with continual agitation via a stir bar. The skin samples were mounted on modified Franz diffusion cell. The dermal side of the skin was exposed to the receptor fluid and the stratum corneum remained in contact with the donor compartment. Accurately weighed Formulation M corresponding to 21 mg of tetracaine and AMETOP corresponding to 26 mg of tetracaine were applied on the donor compartment side of the skin. Aliquots of 400 microliter were withdrawn at time intervals of 0.5 hr, 1 hr, 2 hr, 4 hr, 6 hr, 8 hr, and 24 hr. The same amount of PBS solution was replaced into the receptor compartment after each sampling. The samples were quantified by using HPLC analysis.

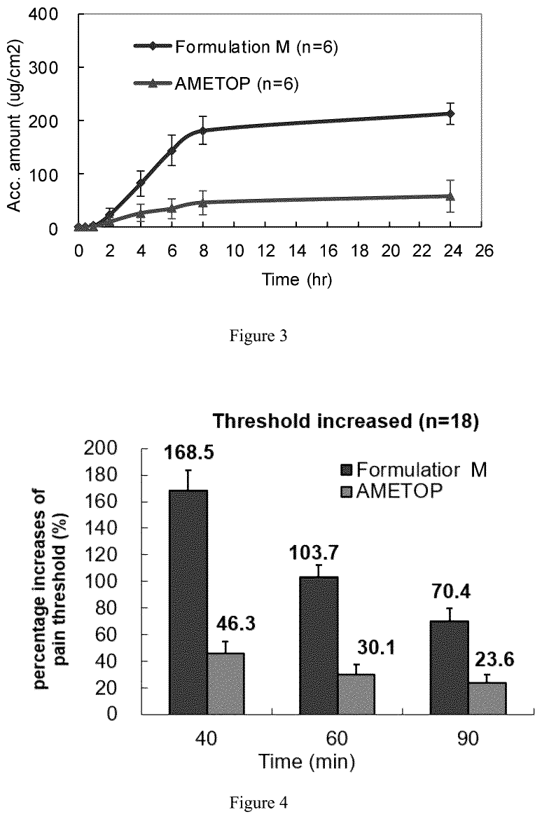

The details of each formulation and the results of the Franz diffusion cell experiments are set out in Table 8 and FIG. 3. With reference to Table 8, it can be seen that Formulation M was more effective at permeating flux through skin when compared to AMETOP Further and surprisingly, Formulation M was also approximately 3.8 times more effective at permeating flux of the tetracaine through skin when compared to commercial product AMETOP. In FIG. 3, the penetration amount of the Formulation M was higher than the AMETOP at each time.

TABLE 8

| | Composition (wt %) | | | | | |
|---|---|---|---|---|---|---|
| | | | | Hydroxy- | | |
| | | | | propyl | | |
| Formu- | Tetracaine | | | cellulose | | Flux |
| lation | (TTC) | Talc | DMSO | (HPC) | water | (μg/cm²/hr) |
| M | 5 | 56 | 35 | 1 | 3 | 53.6 |

| | |
|---|---|
| AMETOP | 14.2 |

### EXAMPLE 9. EFFICACY STUDY FOR TETRACAINE-CLAY COMPOSITE FORMULATION AND COMMERCIAL PRODUCT

The Formulation M and AMETOP were applied to each Sprague Dawley (SD) rat (female rat, obtained from BioLASCO Taiwan Co., Ltd.) respectively. The application time was calculated from the time when the drug was applied. After 30 minutes of application, the drug in the test area was wiped off with gauze to perform von Frey test.

In this experiment, the mechanical sensitivity of the hind paw of rats was examined by Von Frey assay to evaluate the anesthetic effect.

The von Frey hair used in the experiment is a set of nylon needles with different thicknesses. Each von Frey hair has different strengths when puncture, from weak to strong in order of 4, 6, 8, 10, 15, 26, 60 g, a total of 7 nylon needles, which are physical stimuli during testing. Each rat was individually placed in a transparent feeding cage and placed on a stainless steel mesh (6.3×6.3 mm) for an adaptation time of 10 minutes, followed by a strength of von Frey hair. Vertical to the hind paw, the hind paw is pricked from the gap of the stainless steel mesh, and the von Frey hair is maintained in a curved state for 5 seconds, called a stimulus. Each intensity requires 5 stimuli at random locations on the hind paw. The von Frey hairs of different strengths are tested from weak to strong. After completing a strength test, the strength is replaced until the threshold is measured and recorded.

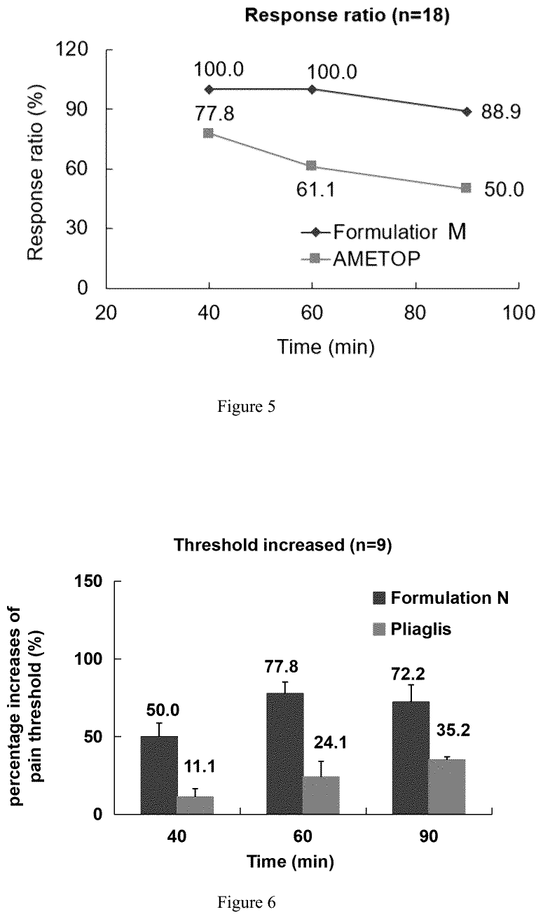

The details of each formulation and the results of the efficacy study are set out in FIG. 4 and FIG. 5. In FIG. 4, it can be seen that Formulation M was more effective at increasing pain threshold when compared to AMETOP. The increased percentage of threshold is 3.6 times that of AMETOP at 40 min. The result showed that the drug of Formula M entered the skin faster than AMETOP. In FIG. 5, the result also showed that 88.9% response ratio in the Formulation M group still had anesthetic effect at 90 min, while 50.0% response ratio in the AMETOP group still had anesthetic effect, indicated that the duration of Formulation M was longer than AMETOP.

### EXAMPLE 10. EFFICACY STUDY FOR TETRACAINE/LIDOCAINE-CLAY COMPOSITE FORMULATION AND COMMERCIAL PRODUCT

The Formulation N, Formulation O and commercial topical lidocaine/tetracaine Pliaglis were applied to each Sprague Dawley (SD) rat (female rat, obtained from BioLASCO Taiwan Co., Ltd.) respectively. The application time was calculated from the time when the drug was applied. After 30 minutes of application, the drug in the test area was wiped off with gauze to perform von Frey test.

In this experiment, the mechanical sensitivity of the hind paw of rats was examined by Von Frey assay to evaluate the anesthetic effect.

The von Frey hair used in the experiment is a set of nylon needles with different thicknesses. Each von Frey hair has different strengths when puncture, from weak to strong in order of 4, 6, 8, 10, 15, 26, 60 g, a total of 7 nylon needles, which are physical stimuli during testing. Each rat was individually placed in a transparent feeding cage and placed on a stainless steel mesh (6.3×6.3 mm) for an adaptation time of 10 minutes, followed by a strength of von Frey hair. Vertical to the hind paw, the hind paw is pricked from the gap of the stainless steel mesh, and the von Frey hair is maintained in a curved state for 5 seconds, called a stimulus. Each intensity requires 5 stimuli at random locations on the hind paw. The von Frey hairs of different strengths are tested from weak to strong. After completing a strength test, the strength is replaced until the threshold is measured and recorded.

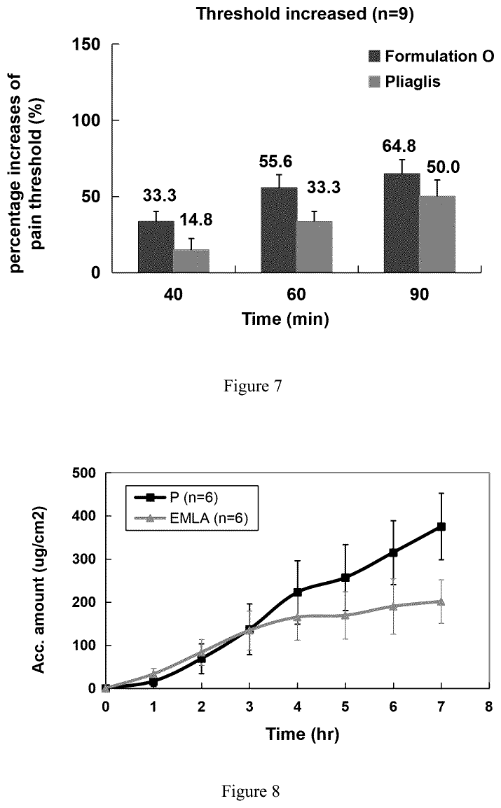

The details of each formulation and the results of the efficacy study are set out in Table 9, FIG. 6, and FIG. 7. In FIG. 6, it can be seen that Formulation N was more effective at increasing pain threshold when compared to Pliaglis. The increased percentage of threshold is 4.5 times that of Pliaglis at 40 min. The result showed that the drug of Formula N entered the skin faster than Pliaglis. In FIG. 7, it can be seen that Formulation O was more effective at increasing pain threshold when compared to Pliaglis. The increased percentage of threshold is 2.3 times that of Pliaglis at 40 min. The result showed that the drug of Formula O also entered the skin faster than Pliaglis.

TABLE 9

| | Composition (wt %) | | | | | | | |
|---|---|---|---|---|---|---|---|---|
| | | | | | | Hydroxypropyl | | |
| | Tetracaine | Lidocaine | | Dicalcium | | cellulose | Transcutol | |
| Formulation | (TTC) | (LDC) | Talc | Phosphate | DMSO | (HPC) | p | Petrolatum |
| N | 7 | 7 | 24 | 32 | 20 | 1 | 9 | — |
| O | 7 | 7 | 24 | 32 | 23 | 1 | — | 6 |

### EXAMPLE 11. SKIN PERMEATION FOR LIDOCAINE-CLAY COMPOSITE FORMULATION AND COMMERCIAL PRODUCT

Formulation P and the commercial topical lidocaine/prilocaine EMLA were tested for permeation through porcine skin using the Franz diffusion cells with a 6 ml receptor well volume. The porcine skin was shaved free of hair, washed with phosphate buffered saline (PBS) and subcutaneous fat was removed. The donor well had an area of 0.785 cm². Receptor wells were filled with isotonic PBS doped. The flanges of the Franz cell were clamped together with uniform pressure using a pinch clamp. Receptor wells of the Franz cells were maintained at 32° C. in a stirring block with continual agitation via a stir bar. The skin samples were mounted on modified Franz diffusion cell. The dermal side of the skin was exposed to the receptor fluid and the stratum corneum remained in contact with the donor compartment. Accurately weighed Formulation P corresponding to 2.925 mg of lidocaine and EMLA corresponding to 2.925 mg of lidocaine were applied on the donor compartment side of the skin. Aliquots of 400 microliter were withdrawn at time intervals of 1 hr, 2 hr, 3 hr, 4 hr, 5 hr, 6 hr, and 7 hr. The same amount of PBS solution was replaced into the receptor compartment after each sampling. The samples were quantified by using HPLC analysis.

The details of each formulation and the results of the Franz diffusion cell experiments are set out in Table 10 and FIG. 8. With reference to Table 10, it can be seen that Formulation P was more effective at permeating flux through skin when compared to EMLA Further and surprisingly, Formulation P was also approximately 1.9 times more effective at permeating flux of the lidocaine through skin when compared to commercial product EMLA. In FIG. 8, the penetration amount of the Formulation P was higher than the EMLA at each time.

TABLE 10

| | Composition (wt %) | | | | LDC Flux |
|---|---|---|---|---|---|
| Formulation | Lidocaine (LDC) | Talc | DMSO | water | (μg/cm²/hr) |
| P | 2.5 | 56 | 34.5 | 7 | 56.8 |

| | |
|---|---|
| EMLA | 29.8 |

### EXAMPLE 12. SKIN PERMEATION FOR PRILOCAINE-CLAY COMPOSITE FORMULATION AND COMMERCIAL PRODUCT

Formulation Q and the commercial topical lidocaine/prilocaine EMLA were tested for permeation through porcine skin using the Franz diffusion cells with a 6 ml receptor well volume. The porcine skin was shaved free of hair, washed with phosphate buffered saline (PBS) and subcutaneous fat was removed. The donor well had an area of 0.785 cm². Receptor wells were filled with isotonic PBS doped. The flanges of the Franz cell were clamped together with uniform pressure using a pinch clamp. Receptor wells of the Franz cells were maintained at 32° C. in a stirring block with continual agitation via a stir bar. The skin samples were mounted on modified Franz diffusion cell. The dermal side of the skin was exposed to the receptor fluid and the stratum corneum remained in contact with the donor compartment. Accurately weighed Formulation Q corresponding to 2.925 mg of prilocaine and EMLA corresponding to 2.925 mg of prilocaine were applied on the donor compartment side of the skin. Aliquots of 400 microliter were withdrawn at time intervals of 1 hr, 2 hr, 3 hr, 4 hr, 5 hr, 6 hr, and 7 hr. The same amount of PBS solution was replaced into the receptor compartment after each sampling. The samples were quantified by using HPLC analysis.

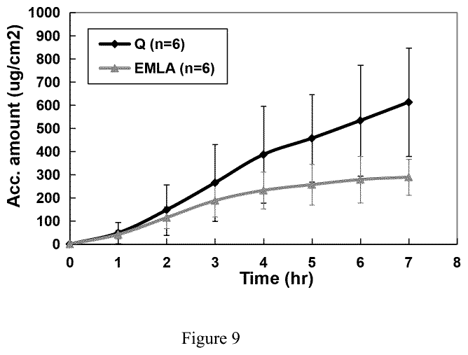

The details of each formulation and the results of the Franz diffusion cell experiments are set out in Table 11 and FIG. 9. With reference to Table 11, it can be seen that Formulation Q was more effective at permeating flux through skin when compared to EMLA Further and surprisingly, Formulation Q was also approximately 2.1 times more effective at permeating flux of the prilocaine through skin when compared to commercial product EMLA. In FIG. 9, the penetration amount of the Formulation Q was higher than the EMLA at each time.

TABLE 11

| | Composition (wt %) | | | | PLC Flux |
|---|---|---|---|---|---|
| Formulation | Prilocaine (PLC) | Talc | DMSO | water | (μg/cm²/hr) |
| Q | 2.5 | 56 | 34.5 | 7 | 92.5 |

| | |
|---|---|
| EMLA | 43.8 |

All the documents mentioned in the present invention are incorporated in the present application by reference to the same extent as if each individual document is specifically and individually indicated to be incorporated by reference. In addition, it should be understood that after reading the contents taught in the present invention, various modifications and changes may be made to the present invention by those skilled in the art, and these equivalents also fall into the scope defined by the claims.

What is claimed:

1. An anesthetic composition for topical application comprising:
(a) therapeutically effective amount of at least one pharmaceutically active agent;
(b) at least one pharmaceutically acceptable solvent vehicle;
(c) at least one clay;
wherein the pharmaceutically active agent is ester type or amide type anesthetic agent.

2. The anesthetic composition of claim 1, wherein the composition is in semi-solid form and store at ambient temperatures.

3. The anesthetic composition of claim 1, wherein the ester type anesthetic agent is in an amount of about 1 to 10% based on the weight of the whole composition.

4. The anesthetic composition of claim 1, wherein the amide type anesthetic agent is in an amount of about 1 to 10% based on the weight of the whole composition.

5. The anesthetic composition of claim 3, wherein the ester type anesthetic agent is in an amount of 5% or 7% based on the weight of the whole composition.

6. The anesthetic composition of claim 4, wherein the amide type anesthetic agent is in an amount of 5% or 7% based on the weight of the whole composition.

7. The anesthetic composition of claim 1, wherein the clay represents about 5 to 60% based on the weight of the whole composition.

8. The anesthetic composition of claim 7, wherein the clay represents 13 to 56% based on the weight of the whole composition.

9. The anesthetic composition of claim 3, wherein the anesthetic agent is selected from the group consisting of benzocaine, chloroprocaine, cyclomethycaine, dimethocaine, piperocaine, propoxycaine, procaine, proparacaine, tetracaine.

10. The anesthetic composition of claim 4, wherein the anesthetic agent is selected from the group consisting of lidocaine, mepivacaine, prilocaine, bupivacaine, ropivacaine and articaine.

11. The anesthetic composition of claim 1, wherein the clay is selected from the group consisting of nanosilicate platelets, montmorillonite, bentonite, mica, laponite, kaolin, talc, hydrotalcite, attapulgite clay, vermiculite, hectorite, saponite, stevensite, beidellite or layered double hydroxides.

12. The anesthetic composition of claim 1 further comprising at least one excipient.

13. The anesthetic composition of claim 1 further comprising a pharmaceutically acceptable solvent vehicle, wherein the carrier is selected from the group consisting of methanol, ethanol, propyl glycol, ethyl acetate, isopropyl alcohol, transcutol P, PEG300, PEG400, glycerol, acetone, lauryl alcohol, oleyl alcohol, cyclomethicone, methylene chloride, benzyl alcohol, acetone, acetic acid, propylene carbonate, dichloromethane, chloroform, 1,4-dioxane, dimethylformamide, dimethyl sulphoxide, toluene, tetrahydrofuran, dodecanol, water or their combination.

14. The anesthetic composition of claim 1 which comprises:
(a) 1 to 10 weight percent of anesthetic agent; and
(b) 5 to 60 weight percent of talc; and
(c) 20 to 90 weight percent of pharmaceutically acceptable vehicle; and/or
(d) 1 to 5 weight percent of HPC.

15. The anesthetic composition of claim 14 comprising 5 weight percent of tetracaine and 56 weight percent of talc.

16. The anesthetic composition of claim 14 comprising 7 weight percent of tetracaine, 7 weight percent of lidocaine, and 24 weight percent of talc.

17. The anesthetic composition of claim 14 comprising 5 weight percent of tetracaine, 56 weight percent of talc, 35 weight percent of DMSO, and 1 weight percent of HPC.

18. The anesthetic composition of claim 14 comprising 7 weight percent of tetracaine, 7 weight percent of lidocaine, 24 weight percent of talc, 32 weight percent of dicalcium phosphate, 20 weight percent of DMSO, 3 weight percent of transcutol p, and 1 weight percent of HPC.

19. The anesthetic composition of claim 14 comprising 7 weight percent of tetracaine, 7 weight percent of lidocaine, 24 weight percent of talc, 32 weight percent of dicalcium phosphate, 23 weight percent of DMSO, 6 weight percent of petrolatum, and 1 weight percent of HPC.

20. A method of administering one or more pharmaceutically active agents to a subject comprising the steps of:
(a) providing the composition of any of claims 1 to 19; and
(b) contacting an area of skin with the anesthetic composition.
